(19) United States
(12) Patent Application Publication
(10) Pub. No.: US 2014/0370499 A1
(43) Pub. Date: Dec. 18, 2014

(54) DEVICE AND METHOD FOR FRAGMENTING POLYMERS AND PARTICLES

(71) Applicant: The Broad Institute, Inc., Cambridge, MA (US)

(72) Inventors: Son Hoang, Dorchester, MA (US); Filip Rege, Belmont, MA (US); Patrick Cahill, Natick, MA (US)

(21) Appl. No.: 14/368,108

(22) PCT Filed: Dec. 20, 2012
(86) PCT No.: PCT/US2012/070946

§ 371 Date: Jun. 23, 2014

Related U.S. Application Data
(60) Provisional application No. 61/579,713, filed on Dec. 23, 2011.

(51) Int. Cl. C12Q 1/68

(52) U.S. Cl. CPC C12Q 1/6806
USPC 435/6.1; 435/309.1

(57) ABSTRACT

The invention features devices for fragmenting polymers or particles in a sample using a centrifuge. In its simplest embodiment, the device includes a body and a valve. The valve, to one side of which a sample is applied, is designed to remain closed until a specified centrifugal force is reached or exceeded. Once open, the valve allows the passage of the sample through a channel in the body. The channel includes one to four fragmenting regions, and the polymers or particles are fragmented as they traverse the fragmenting regions under an applied centrifugal force. The invention further features methods of fragmenting polymers using a device of the invention.

### CROSS-REFERENCE TO RELATED APPLICATIONS

This application claims benefit of U.S. provisional application No. 61/579,713, filed Dec. 23, 2011, which is hereby incorporated by reference.

### BACKGROUND OF THE INVENTION

The present invention relates to the field of biological sample preparation. The sequencing of nucleic acid molecules, such as deoxyribonucleic acid (DNA) and ribonucleic acid (RNA) molecules, facilitates the analysis of genetic information from biological organisms. A nucleic acid molecule is sequenced by determining the order of nucleotide bases within a strand of the molecule. Sequence information can have a powerful impact on biological research and discovery. For example, this information can be used in the development of diagnostic and therapeutic methods for diseases and conditions that are associated with a particular nucleic acid molecule (e.g., a mutated version of the nucleic acid molecule).

A number of different, automated technologies have been developed to assist in the sequencing of nucleic acid molecules. Prior to sequencing, a molecule is typically fragmented to a size that is appropriate for the sequencing instrumentation. Different instruments have different preferred fragment size ranges. Some technologies for fragmentation are costly, prone to contamination, and difficult to implement. There is a need for devices and methods that produce nucleic acid molecule fragments of appropriate size, on a consistent basis, for use in automated sequencing instruments.

### SUMMARY OF THE INVENTION

The invention features devices and methods for use in fragmenting polymers, such as polynucleotides, or particles, such as cells.

In one aspect, the invention features a device for fragmenting polymers or particles in a liquid sample. The device includes a body adapted for use in a centrifuge; a valve; and a channel formed in the body. The channel has a first end through which the sample enters and a second end through which the fragments exit, and one to four fragmenting regions are disposed between the first and second ends. When the valve opens, the sample passes through the valve and channel, and the polymers or particles are fragmented as they traverse the one to four fragmenting regions under a centrifugal force.

In various embodiments, the body further includes a sample compartment, e.g., separated from the channel by the valve, where the sample is substantially retained in the sample compartment until the valve opens. The body is preferably adapted to be supported in a centrifuge tube. Placement of the device in the centrifuge tube may also define a sample compartment, e.g., separated from the channel by the valve, where the sample is substantially retained in the sample compartment until the valve opens. Alternatively, the body is shaped for placement in a centrifuge in the absence of a centrifuge tube.

The valve can open at or above the centrifugal force. Examples of such valves include a frangible barrier, a spring-loaded valve, or a plug that is released from the body. Alternatively, the valve can be electronically controlled, temperature controlled, or time controlled.

The polymers are, for example, polynucleotides or polypeptides, or the particles are, for example, cells, cellular organdies, lipid particles, or micelles that are fragmented as they traverse the one to four fragmenting regions. In particular, polynucleotides can be fragmented on average into pieces ranging from 100 by to 40,000 bp.

In certain embodiments, only one fragmenting region is disposed in the channel. Each fragmenting region can also be a narrowed region of the channel disposed at or adjacent to the second end. For example, the narrowed region has a diameter of between 1 and 1,000 μm. Each fragmenting region can include a plurality of narrowed regions of the channel disposed at or adjacent to the second end.

In other embodiments, the body further includes a compartment to receive fragmented molecules or particles passing through the channel.

The device may also include a filter disposed to contact the fragmented sample after exiting the second end. Examples of filters include a size-exclusion filter and a filter having an affinity reagent for binding a component of the fragmented sample. Multiple filters may be present to collect fragments in a specified size range. The filter may or may not be included within the body of the device.

The device may also include an agent selected from the group consisting of enzymes, solvents, detergents, and biological agents to disrupt particles. Such agents may stored in liquid or dry form in the device, e.g., in a disrupting compartment. A device may also include an acoustic transducer for sonication of the sample, electrodes for delivery an electric field to the sample, a heater or cooler, or a homogenizer having rotary cutting or chopping action. The device may also include a filter to separate debris from particle disruption.

The invention also features a kit including a plurality of any of the devices of the invention and a holder adapted for supporting the plurality of devices in a centrifuge.

In a related aspect, the invention features methods for fragmenting polymers or particles in a liquid sample using a device of the invention. Such methods include providing a device including a body adapted for use in a centrifuge; a valve; and a channel formed in the body and having a first end through which the sample enters and a second end through which the fragments exit, wherein one to four fragmenting regions are disposed between the first and second ends. In addition, either the body further includes a sample compartment, e.g., separated from the channel by the valve, or placement of the device in the centrifuge tube defines a sample compartment, e.g., separated from the channel by the valve. The sample is substantially retained in the sample compartment until the valve opens. The methods further include placing the sample in the sample compartment; centrifuging the device at a centrifugal force, e.g., between 1,000 and 200,000 G; allowing the valve to open or opening the valve, wherein the sample passes through the valve and channel, and the polymers or particles are fragmented as they traverse the one to four fragmenting regions. The method can also be repeated on the same sample.

When the device includes a filter disposed to contact the fragmented sample after exiting the second end, the filter may concentrate the fragmented sample or include an affinity reagent for binding a component of the fragmented sample. When the device includes multiple filters, the method may be employed to collect fragments based on multiple properties. For example, fragments may be separated by size and affinity, two different types of affinity (or lack of affinity), or size between two different size exclusions, e.g., polynucleotide fragments between 500 and 1000 bp.

The method may also include contacting the sample with an agent selected from the group consisting of enzymes, solvents, detergents, and biological agents to disrupt particles. The method may alternatively include disrupting particles in the sample with sonication, electric field, heat, cold, or a homogenizer having rotary cutting or chopping action. In such methods, the device may include a filter or filters to separate debris from particle disruption from the sample to be fragmented.

In certain embodiments, the valve opens at or above the centrifugal force. Examples of such valves include a frangible barrier, a spring-loaded valve, or a plug that is released from the body. Alternatively, the valve is electronically controlled, temperature controlled, or time controlled.

The polymers are, for example, polynucleotides or polypeptides, or the particles are, for example, cells, cellular organelles, lipid particles, or micelles that are fragmented as they traverse the one to four fragmenting regions. In particular, polynucleotides can be fragmented on average into pieces ranging from 100 by to 40,000 bp.

In certain embodiments, only one fragmenting region is disposed in the channel. Each fragmenting region can also be a narrowed region of the channel disposed at or adjacent to the second end. For example, the narrowed region has a diameter of between 1 and 1,000 μm. Each fragmenting region can include a plurality of narrowed regions of the channel disposed at or adjacent to the second end.

The invention provides several advantages. For example, the invention facilitates and improves the fragmentation of components of biological samples, such as nucleic acid molecules and cells. The invention allows fragmentation of polymers or particles with a fast processing time (<1 minute), great ease of use, and reduced material and process costs. In certain examples, the invention achieves a desired level of fragmentation, e.g., length of fragmented nucleic acid molecules, in a single use. The device can also be disposable, leading to decreased risk of sample contamination and increased convenience to the user.

Other features and advantages will be apparent from the following description, the drawings, and the claims.

By “channel” is meant an open volume through which liquid can flow. Channels as present in the devices of the invention are typically enclosed, except at the inlet and outlet, by the body of the device or the body in combination with a centrifuge tube.

By “particle” is meant a material that does not dissolve in the liquid in which it is suspended during the time course of a fragmentation process according to the invention. Exemplary particles include cells (e.g., bacterial, plant, fungal, protist, or animal cells), cellular organelles (e.g., nuclei, mitochondria, ribosomes, and chloroplasts), viruses, lipid particles (e.g., liposomes or lipid droplets in an oil-in-water emulsion), and micelles.

By “polymer” is meant a molecule made up of a plurality of monomers and having a molecular weight of greater than 300 g/mol. Exemplary polymers include nucleic acid molecules, such as DNA, RNA, and RNA/DNA hybrids, which optionally may include nucleotide analogs or other modifications, e.g., modified bases or backbones. Additional polymers include polypeptides, lipids, polysaccharides, and organic polymers (e.g., polyethylene glycol).

By a sample that is “substantially retained” in the sample compartment is meant that the sample is prevented from exiting the channel connected to the sample compartment until the valve is opened. When the valve is placed between the sample compartment and the channel, the valve will prevent the sample from entering the channel until the valve opens. When the valve is placed at the end of the channel, a portion of the sample may (or may not) enter the channel up to the valve, prior to opening. This volume of fluid in the channel is typically small compared to total sample volume. By “valve” is meant a barrier to flow of a liquid.

### BRIEF DESCRIPTION OF THE DRAWINGS

FIGS. 1A-1D are top and side views of a device of the invention.

FIGS. 2A-2C are top and side views of a device of the invention employing a plug.

FIGS. 3A-3D are top and side views of a device of the invention having a filter.

FIGS. 4A-4D are a set of graphs showing the fragmentation of DNA in devices of FIG. 1. FIGS. 4A-4C show fragmentation at various speeds in a device with a fragmenting region having a diameter of 50 μm. FIG. 4D shows fragmentation in a device with a fragmenting region having a diameter of 10 μm.

### DETAILED DESCRIPTION OF THE INVENTION

In general, the invention provides devices and methods for use in fragmenting polymers or particles in liquid samples. The fragmented materials can then be subjected to further analysis in the course of biological research and discovery. For example, fragmented nucleic acid molecules can be subject to sequence analysis using automated sequencing instrumentation. The devices and methods of the invention are described further as follows.

Devices

The invention features devices for fragmenting polymers or particles in a sample using a centrifuge. In its simplest embodiment, the device includes a body and a valve. The valve, to one side of which a sample is applied, is designed to remain closed until a specified centrifugal force is reached or exceeded. Once open, the valve allows the passage of the sample through a channel in the body. The channel includes one to four fragmenting regions, and the polymers or particles are fragmented as they traverse the fragmenting regions under an applied centrifugal force. The valve may be located at the entrance or exit of the channel.

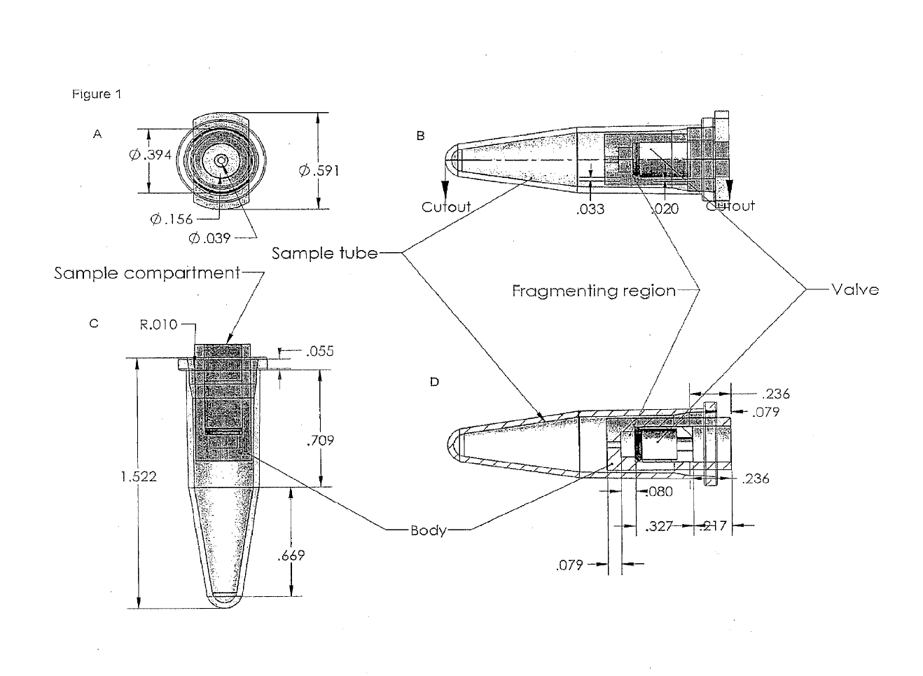

An exemplary device of the invention is shown in FIGS. 1A-1D. This device is designed to fit into a 1.5 mL centrifuge tube. The device includes a sample compartment in the body of the device and a valve, shown here as a relief valve, which opens at or above a specified centrifugal force. FIG. 1A shows a top view of the device placed in a centrifuge tube. The device includes tabs at its top to aid in placement and removal of the device in and from the tube. FIGS. 1B-1D show side views of the device. The device includes a single fragmenting region, which is a narrow channel in this example. Fragmented polymers or particles are collected from the tube when fragmentation is complete.

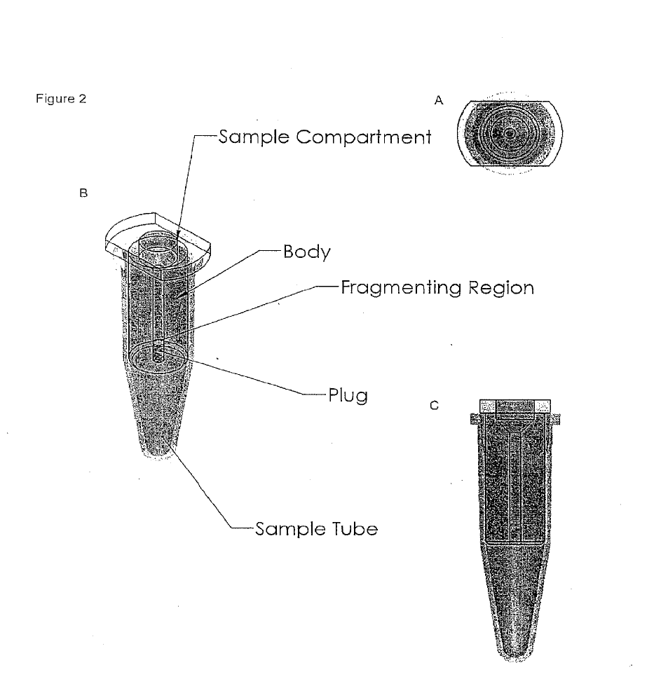

Another exemplary device is shown in FIGS. 2A-2C. This device is also designed to fit into a 1.5 mL centrifuge tube. The device includes a sample compartment in the body of the device and a valve. The valve is a plug that is held in the device by friction or adhesive. At or above a specified centrifugal force, the plug is forced out of the body, allowing fluid to pass through the fragmenting region. FIG. 2A shows a top view of the device placed in a centrifuge tube. The device includes tabs at its top to aid in placement and removal of the device in and from the tube. FIGS. 2B-2C show side views of the device. The device includes a single fragmenting region, which is a narrow channel in this example. Fragmented polymers or particles are collected from the tube when fragmentation is complete.

The device is designed for use in a centrifuge. Exemplary centrifuges are benchtop centrifuges that operate at 1,000-100,000 G. The devices may fit within centrifuge tubes of various sizes, which are typically made of polypropylene or polyethylene. Examples of sizes include those having a volume of 250 μL to 2.0 mL (e.g., 1.5 mL). Larger tubes may also be employed. The exact size and shape of the tube is not critical to the invention but is preferably commensurate with the size of the sample being fragmented. In other examples, the device is formed as an integral unit within a centrifuge tube. The device may be employed with or without a cap or lid. When present, the cap or lid may be separable from the body or centrifuge tube, or it may be physically attached to the body or centrifuge tube.

Body. The body of the device is adapted for use in a centrifuge, with or without being shaped to fit into a centrifuge tube. A channel is formed in the body and is typically enclosed entirely within the body, but also may be formed by placement of the body into a centrifuge tube. The body may include other elements, such as sample or product compartments. A sample compartment, e.g., separated from the channel by a valve, is capable of containing a liquid sample before the valve opens. It will be understood that, when the valve is placed at the exit of the channel, a portion of the sample may fill the channel up to the valve before the valve opens. A product compartment is placed to receive the liquid and fragmented polymers or particles after exiting the channel. In preferred embodiments, the sample and/or product compartments are formed by insertion of the device into a centrifuge tube. In such examples, the device is placed into the centrifuge tube, and the device is sized so that, when properly placed in the tube, the device fits tightly next to the inner wall of the centrifuge tube. Further, in such placement, sample and/or product compartments formed by a combination of the device and the tube are present at the top (sample) and bottom (product) of the tube.

Valves. Any valve capable of opening at or above a specific centrifugal force may be used in the present invention. Examples of such valves include valves that open at or above a specified centrifugal force, such as spring-loaded valves (e.g., ball valves, relief valves, or check valves), plugs, frangible seals (e.g., perforated aluminum foil), and electronically-controlled valves connected to a pressure (or force) sensor or manually operated. These valves are opened either by the force of the sample pressing against the valve or by electronic control from a pressure (or force) sensor or the user. In another embodiment, the valve is an opening having a shape or surface treatment that prevents fluid flow below the specified centrifugal force. Other valves can be time controlled, i.e., open after a specified amount of time, such as after sufficient time to allow the centrifuge to reach or exceed a desired speed. Such time controlled valves can be controlled electronically or be designed to disintegrate at the desired time, e.g., by dissolution or suspension in the liquid sample. In another alternative, the valve is temperature controlled and can be designed to open when a specific temperature, e.g., either above or below ambient, is reached within the centrifuge. Such valves may be electronically controlled by a temperature sensor or by the user monitoring a temperature sensor. Alternatively, the valve may open based on thermal deformation or disintegration, e.g., melting or liquefaction of a thermally reversible gel. Valves may also be electronically controlled and actuated based on any other suitable mechanism. The valve may be formed as an integrated unit with the body of the device or as a separate component. Plugs may also be employed. The plug may be held in the body by friction or adhesive until the specified centrifugal force level is reached or exceeded. In another embodiment, the plug is held in place by tabs integrated or attached to the body, where the tabs break and release the plug at or above a specified centrifugal force. Plugs may be made of any suitable material, such as rubber, other elastomers, gels, and plastics.

Fragmenting regions. Devices of the invention include one to four fragmenting regions within the channel, preferably one. Examples of fragmenting regions include narrowed regions of the channel. For example, a reduction in the transverse dimension (or cross-sectional area) by at least 10, 20, 30, 40, 50, 60, 70, 80, or 90%. Narrowed regions of the channel can have a diameter of 1 to 1000 e.g., 1 to 200 μm. For example, the region can have a diameter of approximately 10, 20, 50, or 89 μm. When more than one fragmenting region is employed, the narrowed regions are arranged in series in the channel and separated from one another by portions of the channel that are not narrowed. For example, the channel can have up to four narrowed regions separated by wider regions. A fragmenting region may also include a plurality of smaller regions arranged in parallel, such as in a mesh. In certain embodiments, e.g., when a single fragmenting region is employed, the region can be placed at or near to the exit of the channel from the body. The length of the fragmenting region can be altered to control the size of fragments; the length range, for example, from 10 μm to 10 cm.

In certain embodiments, the devices may include more than one fluidically separated channel. Multiple channels can be used to allow faster processing of a single sample, as the sample is divided among the channels, or for parallel processing of multiple, different samples. When multiple aliquots of a single sample are employed, sample movement through the channels may be controlled by a single valve, or each channel may have a separate valve. When multiple, different samples are employed, the device (or combination of device and centrifuge tube) typically includes separate sample and product compartments for each sample. In these embodiments, each sample compartment is in contact with a separate valve, which in turn is connected to its respective channel.

Multiple devices may also be employed in connection with a holder for placement in a centrifuge, e.g., in a rotor bucket, and the multiple devices may optionally be packaged together as a kit. Such kits facilitate the parallel processing of multiple samples or multiple aliquots of a single sample in separate devices. Multiple devices that produce differently sized fragments can also be combined in a kit.

The device and its components may be fabricated in any suitable material. Examples of such materials include silicon, glass, polymers (e.g., plastic), and metals. Preferably, the body is formed in a polymer by injection or other molding technique. The fragmenting regions may be fabricated in plastic or may be laser-drilled sapphire/ruby or steel.

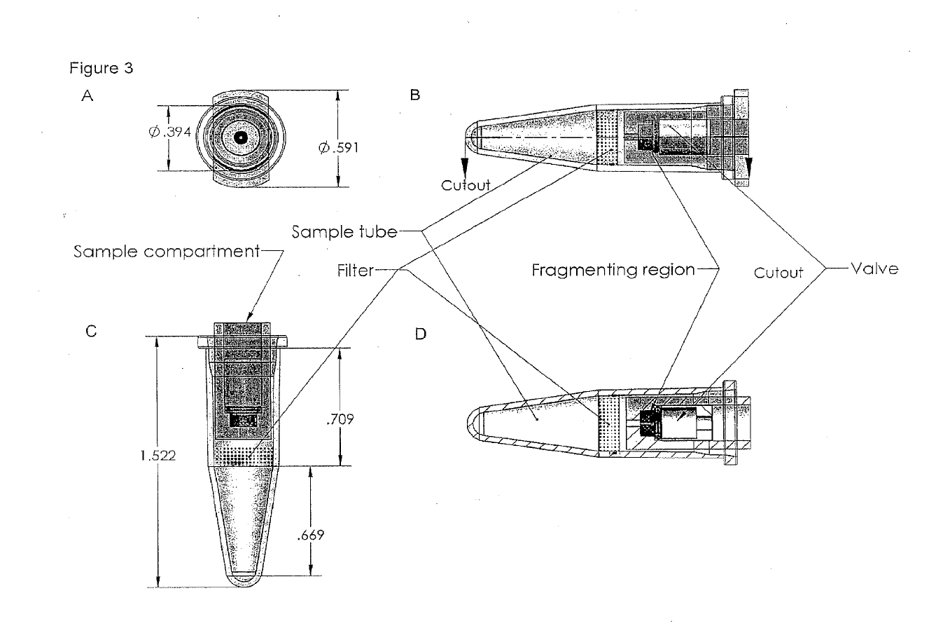

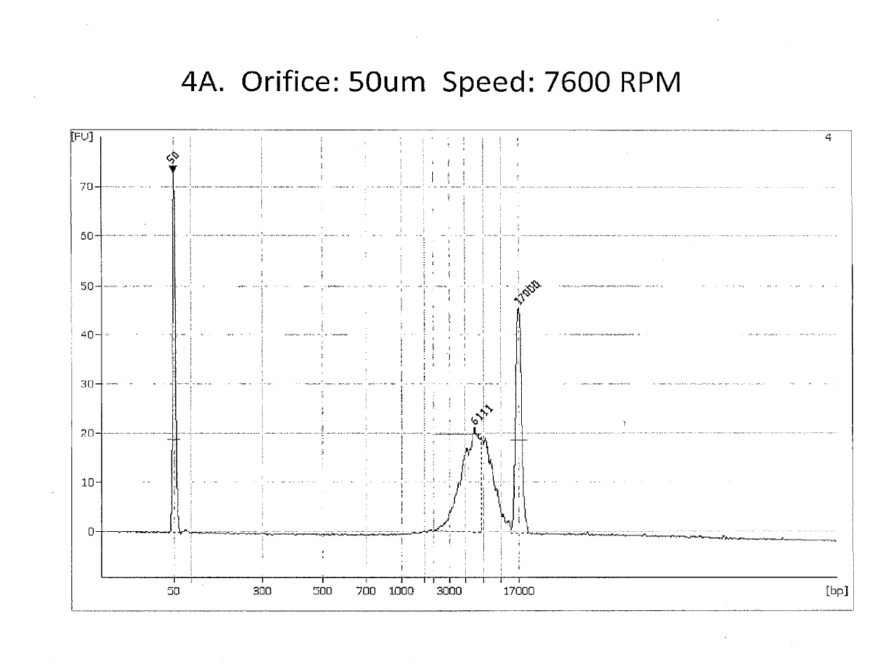

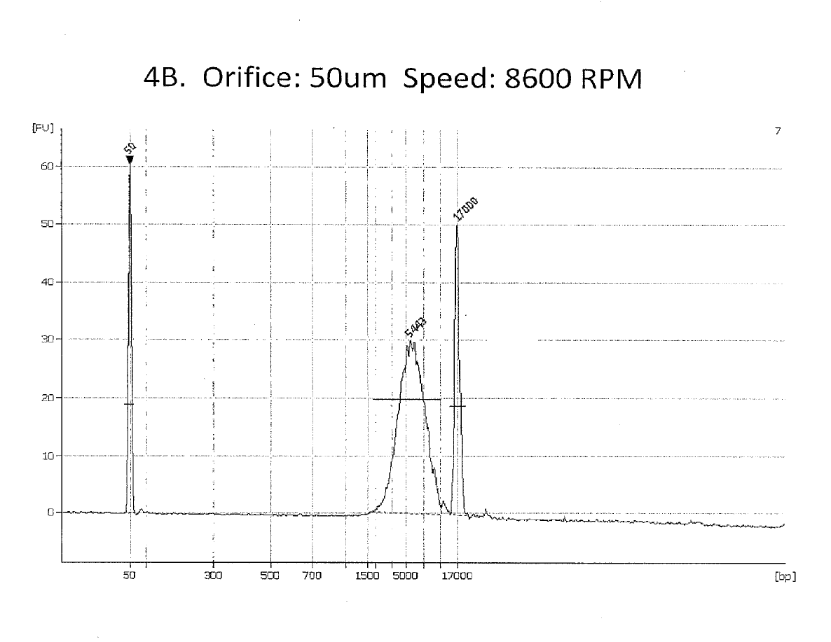

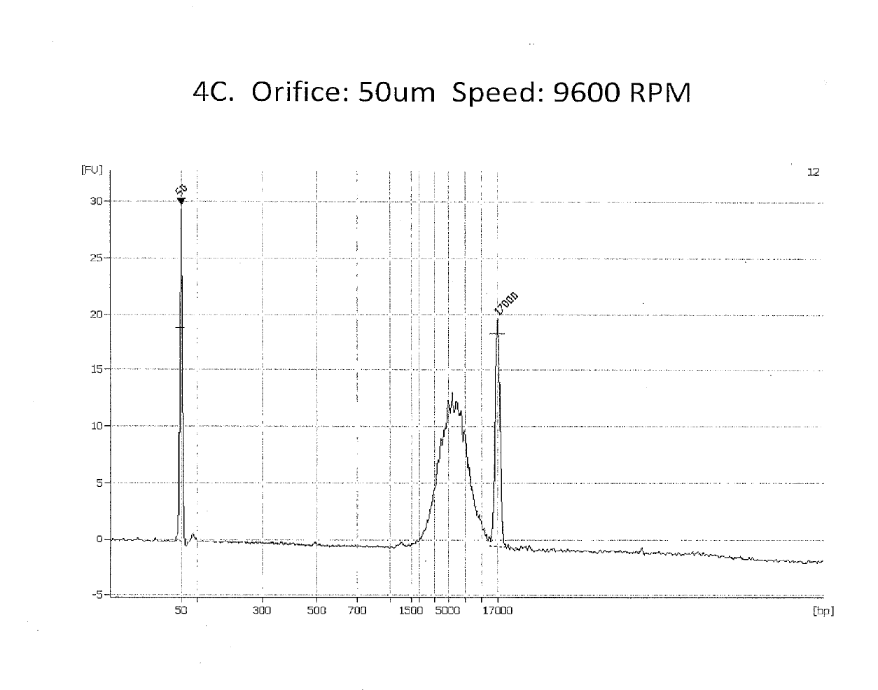

Additional Components. Devices of the invention may include additional components, either as part of the body or as separate pieces to be placed in a centrifuge tube. In one embodiment, a device includes one or more filters, e.g., size exclusion or affinity-based filters. Such filters may be included in the body, e.g., after the second end of the channel, or in a centrifuge tube. Depending on the desired output, the filters may allow solvent and/or a portion of the fragments to pass. Thus, the filters may be employed to concentrate the sample after fragmentation or to select a range of sizes of fragments (or multiple ranges if multiple filters are employed). Filters will be physically spaced apart from the second end or other filters to allow sample to accumulate in the space. An exemplary device of the invention is shown in FIGS. 3A-3D. This device is designed to fit into a 1.5 mL centrifuge tube, although it can be scaled to fit in tubes of other sizes. The device includes a sample compartment in the body of the device and a valve, which opens at or above a specified centrifugal force, and a size exclusion filter. FIG. 3A shows a top view of the device placed in a centrifuge tube. The device includes tabs at its top to aid in placement and removal of the device in and from the tube. FIGS. 3B-3D show side views of the device. The device includes a single fragmenting region, which is a narrow channel in this example.

Devices of the invention may also include components for disruption of particles, e.g., cells. A component to disrupt particles may be part of the body or a separate piece in a centrifuge tube. A fragmenting region of a device may be employed to fragment particles and placed in series with one or more fragmenting regions to fragment polymers or smaller particles. In one embodiment, at least two fragmenting regions are present in series; the one closest to the first end fragments particles, and subsequent fragmenting regions fragment polymers or particles released by the first region. Regions may be designed to reduce the fragments produced in a stepped manner. In another embodiment, the device includes chemical agents to disrupt particles. Exemplary chemical agents include enzymes (e.g., lysozyme, lysostaphin, zymolase, cellulase, mutanolysin, glycanases, proteases, and mannase), solvents (e.g., distilled water or organic solvents such as acetone), detergents (e.g., CHAPS, Triton X, and SDS), and biological agents (e.g., viruses such as bacteriophage). Such agents may be stored in liquid or dry form in a sample compartment or in a separate disrupting compartment. The disruption compartment may be opened by a valve as described herein or by dissolution or suspension in a liquid sample. A device may include an acoustic transducer for sonication. Disruption may also occur by pressurizing the sample and then rapidly releasing the pressure; in such cases, the device and valve would be able to withstand the elevated pressure. Disruption may occur by thermal shock, and the device may include an element, e.g., a resistive heater or Peltier, to raise or lower the temperature. An electric field may also be used to disrupt particles, with the device including electrodes for delivery of the field. Devices may also include a homogenizer having rotary cutting and/or chopping action using compact blades or paddles, e.g., turning at speeds of 10,000 to 30,000 rpm. Finally, the device may include beads, which can be shaken with the sample to disrupt particles. The device may also further include filters to separate debris from disruption prior to the sample entering the channel. The device may also include solid substrates, e.g., beads or surfaces of the body or centrifuge tube, to bind polymers or smaller particles of interest to allow rinsing of the device after disruption and prior to fragmentation.

Methods

Polymers or particles are fragmented by passing through a device of the invention under an appropriate centrifugal force. The average size and distribution of fragments is determined by factors including the size of the starting polymer or particle, the centrifugal force, and the number and nature of the fragmenting regions.

In the process, a liquid sample containing the polymers or particles is placed in a sample compartment in the device (or in a combination of the device and centrifuge tube, as described above). The sample is then subjected to a centrifugal force in a centrifuge. At or above a specified force, the valve opens and allows the sample to pass through the channel within the body of the device. When the polymers or particles traverse the fragmenting region (or regions), they are subjected to an increased force that results in fragmentation. In certain embodiments, the applied centrifugal force is between 1,000 and 200,000 G, e.g., between 1,000 and 16,000 G. The sample can also be subjected to fragmentation multiple times, e.g., for a total of 2 to 8 passes through the device.

The methods may be employed with a variety of polymers, e.g., polymers such as polynucleotides (e.g., DNA, such as genomic DNA, or RNA), polysaccharides, lipids, and proteins. The methods are particularly advantageous for fragmenting polynucleotides, e.g., prior to sequencing. Such polynucleotides can be fragmented to average sizes of approximately 100 to 40,000 bp, e.g., 500 bp, 1,000 bp, 3,000 bp, 5,000 bp, or 10,000 bp. It will be understood that the process will produce fragments having a range of sizes. Preferably, the distribution is between 3000 by to 10,000 bp. In addition to preparation for sequencing, polymers or particles can be fragmented prior to being subjected to analysis, e.g., by mass spectrometry or chromatography, or to reduce size heterogeneity. Polymers can be fragmented to average sizes of, for example, 500 g/mol. The methods of the invention may also be used to fragment particles, such as cells, organelles, viruses, lipid particles (e.g., liposomes or lipid droplets in an oil-in-water emulsion), and micelles, to release the internal components of the particles, or to produce smaller or more uniform particles.

Particles, e.g., cells, may be disrupted in a device of the invention, and the released polymers and particles, e.g., organelles, may then be fragmented by one or more fragmenting regions. In one embodiment, at least two fragmenting regions are present in series; the one closest to the first end fragments particles, and subsequent fragmenting regions fragment polymers or particles released by the first region. Regions may be designed to reduce the fragments produced in a stepped manner. In another embodiment, particles are disrupted in the sample compartment or in a disrupting compartment separated from the sample compartment. In these embodiments, disruption may occur by any known method. For example, particles may be subjected to various chemical treatments, including enzymes (e.g., lysozyme, lysostaphin, zymolase, cellulase, mutanolysin, glycanases, proteases, and mannase), solvents (e.g., distilled water or organic solvents such as acetone), detergents (e.g., CHAPS, Triton X, and SDS), and biological agents (e.g., viruses such as bacteriophage). When chemical methods are employed, the agent may be added to the device before or after the sample. Such agents may also be stored in the device, e.g., in liquid or dry form. Such agents may be stored in a compartment that is opened by a valve, as described herein. Other methods for disruption include sonication, electric field, thermal shock (e.g., freezing and thawing), homogenizer having rotary cutting and/or chopping action using compact blades or paddles, e.g., turning at speeds of 10,000 to 30,000 rpm, and beads, which are shaken with the sample. Disruption may also occur by pressurizing the sample and then rapidly releasing the pressure. Disruption may occur inside or outside of a centrifuge. The particular mechanism for disruption will be selected on the basis of the particles being employed, as known to one skilled in the art.

After fragmentation, the sample may be subject to size or affinity selection and/or concentration. The body may include filters, e.g., for size exclusion or affinity-based filtration, that retain fragments of interest or allow fragments of interest to pass. For concentration, a molecular weight cut off for the filter will allow the solvent pass but not the fragments of interest. For size selection, one or more filters, e.g., membranes, may be employed to separate fragments of a selected size range from those having higher and lower molecular weights. This process may also be employed to obtain several size-separated fractions from a sample. When multiple size exclusion filters are employed, the filters will exclude successively smaller fragments, with the largest fragments retained closest to the second end. Affinity may be used to bind fragments of interest. Affinity reagents are known in the art, including antibodies, aptamers, ligands, receptors, and oppositely charged substrates (e.g., positively charged polymers).

The amount of time that a sample is subjected to centrifugal force is preferably less than 1 minute. The time required to process a sample may depend on the concentration of the polymer or particle being fragmented. The time employed will typically be sufficient to result in the passage of the entire sample through the device. Alternatively, only a portion of the sample is passed through the device. Typical sample volumes are between 5 and 200 μL. The concentration of the polymer or particle in the sample is also typically between 1 ng/μL to 50 μg/μL. The longer the polymer chains, the bigger the molecular weight and the sample will be more concentrated, given the same amount of volume. Higher concentrations may lead to clogging of the channel, depending on the nature of the polymer or particle. The liquid in which the sample components are dissolved or suspended will typically be aqueous (e.g., TE (10 mM Tris, 1 mM EDTA, pH 8.0) or water), but non-aqueous solvents can be employed, especially for fragmenting lipids or organic polymers. Additives, such as glycerol, oil and PEG, can also be included in the sample, e.g., to increase viscosity.

### EXAMPLE

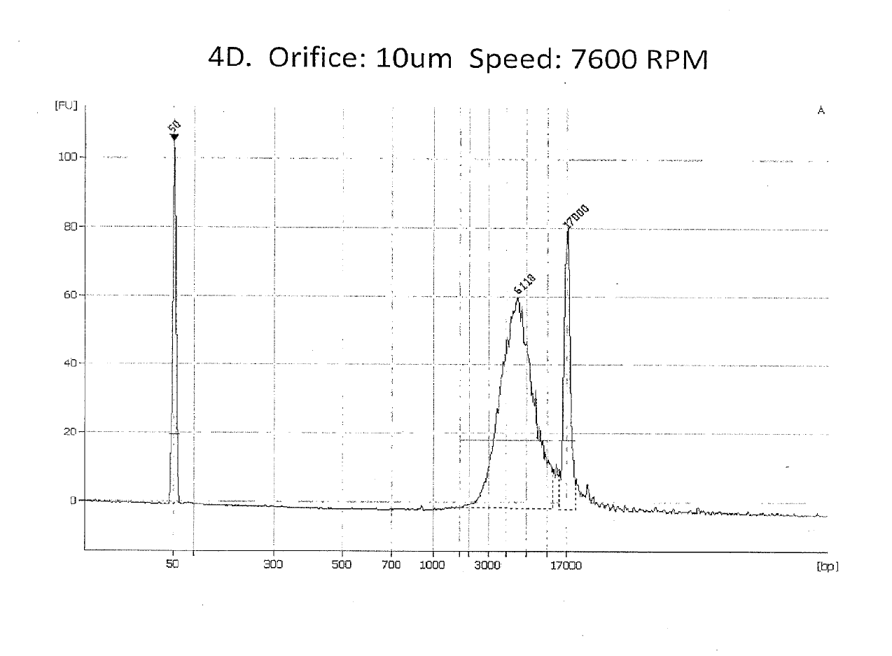

In this example, double stranded mouse genomic DNA (Promega) was diluted to 50 ng/μL in TE buffer. Fifty microliters of sample were placed in the sample compartment of a device illustrated in FIGS. 1A-1D. The device was fitted in a 1.5 mL Eppendorf centrifuge tube. The tube was then centrifuged in an Eppendorf Tabletop Centrifuge 5415D. The sizes of fragments produced were then determined using an Agilent 2100 Bioanalyzer. FIGS. 4A-4D show the results of the fragmentation using devices with fragmenting regions having diameters of 10 μM (FIG. 4D) or 50 μm (FIGS. 4A-4C). As is shown in the figures, the initial sample was approximately 17,000 by long and was fragmented into smaller fragments.

Other embodiments are in the claims.

What is claimed is:

1. A device for fragmenting polymers or particles in a liquid sample, the device comprising a body adapted for use in a centrifuge;
a valve; and
a channel formed in the body and having a first end and a second end, wherein one to four fragmenting regions are disposed between the first and second ends,
wherein the valve opens thereby allowing the sample to pass through the valve and channel, and the polymers or particles are fragmented as they traverse the one to four fragmenting regions under a centrifugal force.

2. The device of claim 1, wherein (i) the body further comprises a sample compartment, and the sample is substantially retained in the sample compartment until the valve opens; (ii) the body is adapted to be supported in a centrifuge tube, (iii) placement of the device in the centrifuge tube defines a sample compartment, and the sample is substantially retained in the sample compartment until the valve opens; (iv) the body further comprises a compartment to receive fragmented molecules or particles passing through the channel; or (v) the body is shaped for placement in a centrifuge in the absence of a centrifuge tube.

3-4. (canceled)

5. The device of claim 1, wherein the valve: (i) opens at or above the centrifugal force; (ii) is a frangible barrier or a spring-loaded valve; (iii) is a plug that is released from the body at or above the centrifugal force; or (iv) is electronically controlled, temperature controlled, or time controlled.

6-8. (canceled)

9. The device of claim 1, wherein the polymers are polynucleotides or polypeptides, or the particles are cells, cellular organelles, lipid particles, or micelles that are fragmented as they traverse the one to four fragmenting regions.

10. The device of claim 9, wherein the polynucleotides are fragmented on average into pieces ranging from 100 by to 40,000 bp.

11. (canceled)

12. The device of claim 1, wherein each fragmenting region is a narrowed region of the channel disposed at or adjacent to the second end, and the narrowed region optionally has a diameter of between 1 and 1,000 μm.

13. (canceled)

14. The device of claim 1, wherein each fragmenting region comprises a plurality of narrowed regions of the channel disposed at or adjacent to the second end.

15. (canceled)

16. The device of claim 1, further comprising a filter disposed to contact the fragmented sample after exiting the second end, wherein optionally (i) the filter is a size-exclusion filter, (ii) the filter comprises an affinity reagent for binding a component of the fragmented sample, (iii) the device further comprises at least an additional filter wherein the filters are disposed to collect fragments in a specified size range, or (iv) the filter is within the body.

17-20. (canceled)

21. The device of claim 1, further comprising an agent selected from the group consisting of enzymes, solvents, detergents, and biological agents to disrupt particles, wherein optionally (i) the agent is stored in liquid or dry form in the device, (ii) the device further comprises a disrupting compartment in which the agent is stored, or (iii) the device further comprises a filter to separate debris from particle disruption.

22-23. (canceled)

24. The device of claim 1, further comprising an acoustic transducer for sonication of the sample, electrodes for delivery an electric field to the sample, a heater or cooler, or a homogenizer having rotory cutting or chopping action.

25-26. (canceled)

27. A device comprising a device of claim 1 placed in a centrifuge tube.

28. A kit comprising a plurality of devices of claim 1 and a holder adapted for supporting the plurality of devices in a centrifuge.

29. A method for fragmenting polymers or particles in a liquid sample, said method comprising the steps of:
a) providing a device comprising:
(i) a body adapted for use in a centrifuge;
(ii) a valve; and
(iii) a channel formed in the body and having a first end and a second end, wherein one to four fragmenting regions are disposed between the first and second ends,
wherein either the body further comprises a sample compartment, or placement of the device in the centrifuge tube defines a sample compartment
wherein the sample is substantially retained in the sample compartment until the valve opens;
b) placing the sample in the sample compartment;
c) centrifuging the device at a centrifugal force; and
d) allowing the valve to open or opening the valve, wherein the sample passes through the valve and channel, and the polymers or particles are fragmented as they traverse the one to four fragmenting regions.

30. The method of claim 29, wherein the valve: (i) opens at or above the centrifugal force, (ii) is a frangible barrier or a spring-loaded valve, (iii) is released from the body at or above the centrifugal force, or (iv) is electronically controlled, temperature controlled, or time controlled.

31-33. (canceled)

34. The method of claim 29, wherein the polymers are polynucleotides or polypeptides or the particles are cells that are fragmented as they traverse the one to four fragmenting regions.

35. The method of claim 34, wherein the polynucleotides are fragmented on average into pieces ranging from 100 bp to 40,000 bp.

36. (canceled)

37. The method of claim 29, wherein each fragmenting region is a narrowed region of the channel disposed at or adjacent to the second end, and optionally the narrowed region has a diameter of between 1 and 1,000 μm.

38. (canceled)

39. The method of claim 29, wherein each fragmenting region comprises a plurality of narrowed regions of the channel disposed at or adjacent to the second end.

40. The method of claim 29, wherein the centrifugal force is between and 1,000 and 200,000 G.

41. The method of claim 29, wherein steps b)-d) are repeated on the same sample.

42. The method of claim 29, wherein the device further comprises a filter disposed to contact the fragmented sample after exiting the second end, and optionally (i) the filter concentrates the fragmented sample, (ii) the filter comprises an affinity reagent for binding a component of the fragmented sample, or (iii) the device further comprises at least an additional filter wherein the filters are disposed to collect fragments in a specified size range.

43-45. (canceled)

46. The method of claim 29, further comprising contacting the sample with an agent selected from the group consisting of enzymes, solvents, detergents, and biological agents to disrupt particles.

47. The method of claim 29, further comprising disrupting particles in the sample with sonication, electric field, heat, cold, or a homogenizer having rotory cutting or chopping action, and optionally the device further comprises a filter to separate debris from particle disruption from the sample to be fragmented.

48. (canceled)
